(19) United States
(12) Patent Application Publication
(10) Pub. No.: US 2006/0083762 A1
(43) Pub. Date: Apr. 20, 2006

(54) Uses of compositions comprising electrophilic monomers and micro-particles or nanoparticles

(76) Inventors: Gaelle Brun, Paris (FR); Aude Livoreil, Paris (FR); Luc Gourlaouen, Asnieres (FR); Gabin Vic, Venette (FR); Franck Giroud, Clichy (FR); Isabelle Rollat-Corvol, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,286

(22) Filed: Oct. 13, 2005

Related U.S. Application Data
(60) Provisional application No. 60/646,485, filed on Jan. 25, 2005.

(30) Foreign Application Priority Data
Oct. 13, 2004 (FR) 04 10806

(51) Int. Cl. A61K 8/81

(52) U.S. Cl. 424/401; 424/70.16; 977/926

(57) ABSTRACT

The present disclosure relates to methods for treating keratin materials, including keratin fibers such as the hair, of a composition comprising, in a cosmetically acceptable medium, at least one electrophilic monomer and microparticles or nanoparticles.

This application claims the benefit of U.S. Provisional Application No. 60/646,485, filed Jan. 25, 2005, the contents of which are incorporated herein by reference. This application also claims benefit of priority under 35 U.S.C. § 119 to French Patent Application No. 04 10806, filed Oct. 13, 2004, the contents of which are also incorporated by reference.

The present disclosure provides uses of and methods for using compositions based on in situ polymerizable monomers, a cosmetically acceptable medium and microparticles or nanoparticles, for the cosmetic treatment of keratin materials, for example, keratin fibers such as hair, and also novel compositions for the cosmetic treatment of keratin materials.

Numerous styling products exist for giving hair volume. One drawback associated with these products, which are typically based on film-forming polymers, is that the cosmetic effect may end with the first shampoo wash.

Permanent-waving treatments of keratin fibers are also known. These treatments utilize a reducing agent and an oxidizing agent, and require the placing of the hair under mechanical tension using rolling equipment in order to impart a shape.

These processes may make it possible to increase the volume of the head of hair, but at the same time may have the drawback of modifying the level of curliness of the hair and of degrading the feel of the fiber.

Thus, there remains a need for compositions for increasing the volume of the head of hair without modifying the shape or feel of the hair, while at the same time being shampoo-fast.

The present disclosure provides novel compositions for overcoming one or more of these drawbacks.

The present inventors have discovered, surprisingly, that by using electrophilic monomers as described in Patent Application No. FR 2 840 208 and microparticles or nanoparticles, it is possible to give a hairstyle volume without impairing the feel or shape of the hair, without degrading the fiber and/or without making the hair adhere together. In addition, these cosmetic properties may withstand several shampoo washes. Further, in addition to the volume of hair, certain particles also make it possible to give the head of hair long-lasting sheen, body, mass, and optical effects.

The present inventors have also discovered, surprisingly, that by applying a composition based on such monomers and on microparticles or nanoparticles to the head of hair, a long-lasting coat covering the hair is formed in situ.

The present disclosure further relates to the use, for treating keratin materials, including keratin fibers such as the hair, of a composition comprising, in a cosmetically acceptable medium, at least one electrophilic monomer and microparticles or nanoparticles.

The disclosure also provides cosmetic processes for treating keratin materials, including keratin fibers such as the hair, using the compositions disclosed heren.

Also disclosed herein are cosmetic compositions comprising, in a cosmetically acceptable medium, at least one electrophilic monomer and microparticles or nanoparticles other than particles of gold or silver.

In some embodiments, the microparticles or nanoparticles in the compositions are exclusively metallic, for example, chosen from aluminum, copper, iron, zinc, tin, manganese, and zirconium. In other embodiments, the microparticles or nanoparticles in the compositions are not exclusively metallic.

In addition, the present disclosure relates to kits comprising a first composition comprising at least one electrophilic monomer (present, for example, in an amount ranging from 0.5 to 50% of the weight of the first composition) and optionally at least one anionic and/or free-radical polymerization inhibitor (present, for example, in an amount ranging from 10 ppm to 5% of the weight of the first composition), and also a second composition comprising, in a cosmetically acceptable medium, microparticles or nanoparticles (present, for example, in an amount independently ranging from 0.001 to 5% of the weight of the second composition).

Other aspects of the invention will become apparent on reading the description and the examples that follow.

As used herein, the term “keratin materials” includes, but is not limited to, keratin fibers, such as hair.

As used herein, the term “nanoparticle” means any particle whose elementary size ranges from 1 to 999 nm, and the term “microparticle” means any particle whose elementary size ranges from 1 to 300 μm.

The nanoparticles or microparticles may be in the form of spheres, needles, flakes, platelets, tubes, fibers, cubes, prisms, or may have irregular forms.

As used herein, the term “particle size” means the distance between the two most distant points of the particle.

Nanoparticles and microparticles that may be used include, but are not limited to electroluminescent semiconductive nanoparticles (or quantum dots), nanofibrils, microfibrils, microplatelets, latices, nanotubes, adhesive microobjects, and expandable particles.

The particles may be mineral, organic, or mixed.

In some embodiments, the particles are mineral. Mineral particles include:
metallic particles. As used herein, the term “metallic particle” means a particle formed from metals chosen from alkaline-earth metals, transition metals, rare-earth metals, and alloys thereof. Examples include aluminum, copper, cadmium, selenium, silver, gold, indium, iron, platinum, nickel, molybdenum, silicon, titanium, tungsten, antimony, palladium, zinc, tin, and alloys thereof. In some embodiments, the particles are formed from metals chosen from gold, silver, palladium, platinum, cadmium, selenium, and alloys thereof. The metallic particles may be organomodified metallic nanoparticles bearing at their surface a self-assembled monolayer of organosulfur compounds as described in Patent Application No. FR 2 838 052.
oxides. Examples include titanium oxide, zinc oxide, cerium oxide, zirconium oxide, aluminum oxide, and bismuth oxychloride.
carbides, nitrides, borides, sulfides, and hydroxides.
mineral salts. Examples include barium sulfate, calcium carbonate, calcium sulfate, calcium phosphate, and magnesium hydrogen carbonate.

Mineral particles that may be used include clays, silicates, alumina, silica, kaolin, and hydroxyapatite.

The particles may also be organic. When the particle is of organic nature, it is generally an organic polymer. Such polymers may be in glassy form, i.e., having a glass transition temperature higher than or significantly higher than room temperature or the working temperature (for example the temperature of the human body), and/or be crosslinked.

The glass transition temperature of the organic polymers that may be used for a solid phase may be greater than or equal to 40° C., such as greater than or equal to 60° C., or ranging from 80° C. to 200° C.

Organic polymers useful herein include, but are not limited to, polystyrene, poly(vinyl acetate), poly (α-methylstyrene), poly(acrylamide), poly(acrylonitrile), poly(vinyl chloride), copolymers based on styrene and on C₁-C₄ alkyl (meth)acrylate, copolymers based on styrene and on acrylamide, copolymers based on styrene and on acrylonitrile, copolymers based on styrene and on vinyl acetate, copolymers based on acrylamide and on C₁-C₄ alkyl (meth)acrylates, copolymers based on acrylonitrile and on C₁-C₄ alkyl (meth)acrylate, copolymers based on acrylonitrile and on acrylamide, terpolymers based on styrene, on acrylonitrile and on acrylamide, poly(methyl methacrylate), poly(ethyl methacrylate) and styrene/butadiene, styrene/acrylic acid, styrene/vinylpyrrolidone, and butadiene/acrylonitrile copolymers.

Organic microparticles and nanoparticles useful herein include, but are not limited to:
nylon powders, for example the powder sold under the name “Orgasol 2002 ED NAT COS” by the company Atochem,
polyethylene powders, for example the powder sold under the name “Coathylene HA 1681” by the company Plast Labor,
poly-β-alanine powders,
polyfluorinated powders, such as polytetrafluoroethylene powders, for example the powder sold under the name “MP 1400” by the company Dupont de Nemours,
acrylic copolymer powders, such as the powders sold under the name “Polytrap Q5 6603” by the company Dow Chemical,
polystyrene powders, such as the powders sold under the name “Polysphere 3000 SP” by the company Presperese,
polyester powders,
expanded microspheres made of thermoplastic material, for example the product sold under the name “Expancel 551 DE” by the company Expancel,
silicone resin microbeads (for example Tospearls from the company Toshiba),
metal soaps derived from organic carboxylic acids having from 8 to 22 carbon atoms (e.g., from 12 to 18 carbon atoms), for example zinc stearate, magnesium stearate, lithium stearate, zinc laurate, and magnesium myristate,
powders of hydrophilic polymers, which are of synthetic origin, for instance polyacrylates, for example the product sold under the name “Micropearl M 100” by the company Matsumoto,
acrylic polyamides, such as those sold by the company Oris,
insoluble polyurethanes, such as the product sold under the name “Plastic Powder D 800” by the company Toshnu,
porous cellulose microspheres, and
PTFE (polytetrafluoroethylene) microparticles or nanoparticles.

The particles may be treated by coating or grafting to obtain mineravorganic mixed particles.

The particles may also be compounds that have been made hydrophilic by chemical grafting or coating using materials such as chitosan, titanium dioxide, silica, and hydrophilic polymers, for example, sulfonic polyesters and polyquaternary ammoniums.

Hydrophobic pulverulent compounds derived from pulverulent compounds of either hydrophobic or hydrophilic nature may also be chosen. Hydrophilic pulverulent compounds may be made hydrophobic by chemical grafting or coating with products such as silicones, amino acids, metal soaps, fluorinated derivatives, mineral oils, lecithin, isopropyl triisostearoyl titanate, polyethylene, collagen and derivatives thereof, and polyacrylates.

Examples include silica microbeads coated with polymethylhydrogenosiloxane, sold under the trade name “Silica SI SB 700” by Miyoshi, or sericite coated with methicone/hydrogenated egg oil, sold under the trade name “Sericite SNI S100” by Miyoshi.

In some embodiments, the particles may be chosen from adhesive particles.

The adhesive polymers may be immobilized on the surface of the particles via covalent chemical bonds (grafting) or via weak physicochemical interactions such as hydrophobic interactions, hydrogen bonding, and Van der Waals forces (adsorption).

The adhesive nature of an organic polymer is generally associated with its glass transition temperature. A necessary but insufficient condition for a polymer to be adhesive is a glass transition temperature (Tg) significantly below room temperature or the working temperature. The adhesive organic polymers used for the preparation of the microobjects of the present disclosure may, in some embodiments, have a glass transition temperature of less than or equal to 10° C., for example, less than or equal to 0° C.

The chemical nature of the adhesive organic polymers is not a determining factor herein, provided that the polymer deposit has adhesive and/or self-adhesive characteristics as described above. These adhesive polymers may or may not be crosslinked. Examples of adhesive polymers are listed in the following patent applications describing adhesive particles or polymers: WO 98/38969 and FR 2 833 960 (self-adhesive cationic or amphoteric polyurethanes), and FR 2 833 959 (self-adhesive cationic or amphoteric free-radical polymers).

In some embodiments, the particles may also be chosen from nanotubes.

The nanotubes may comprise at least one element belonging to groups IIA, IIIA, IVA, VA, VIII, IB, IIB, IIIB, VIB, or VIIB of the Periodic Table, such as group IVA, for example, carbon.

As used herein, the term “nanotubes” means nanoobjects whose atomic or molecular organization gives the nanostructure a tube shape. These nanotubes may be single-walled or multi-walled. The diameter of nanotubes conventionally ranges from 1 to 300 nm and the length from 10 nm to 10 mm.

Examples of constituent elements of nanotubes as described herein include carbon, silicon, tungsten, silver, gold, boron, zinc, platinum, magnesium, iron, cerium, and aluminium.

When one of the constituent elements of the nanotubes is carbon, the nanotubes may be totally or partially comprised of organic molecules. Examples of organic molecules include diacetylenic phospholipids, glutamates, long-chain diamides, glucophospholipids, and alkylphenylglucopyranosides.

In some embodiments, the skeleton of the nanotubes comprises solely carbon atoms. Carbon-based nanoforms such as nanotubes are conventionally obtained by sublimation of graphite at very high temperature using an electric arc. The carbon nanotubes may be formed from a single plane of graphene, known as single-wall nanotubes (SWNT). The graphene planes may be rolled up in zigzag, gap or chiral forms. The nanotubes may also comprise several tubes “slotted” into each other, known as multi-wall nanotubes (MWNT).

In some embodiments, in order to obtain optimum dissolution or exfoliation of the carbon nanotubes in the cosmetic medium, the surface of the nanotubes is functionalized.

As used herein, the term “functionalized” means the presence of functional groups that can physically or chemically interact with each other or with the external medium.

Any reaction mechanism may be used to functionalize the graphene planes constituting the carbon nanotubes. For example, functionalization of the carbon nanotubes may be performed using nucleophilic substitution, electrophilic substitution, free-radical substitution, addition, elimination, rearrangement, oxidation, reduction, acid-base reaction, electrochemical reaction, or photochemical reaction mechanisms.

Functions that may be grafted onto the surface of the graphene planes constituting the carbon nanotubes include carboxylic groups. This functionalization is described in the article “Solution Properties of Single Walled Carbon Nanotube,” J. Chen et al. (Science 1998, vol. 282, No. 5396, pages 95-98).

Dissolution of nanotubes may also be done in a polar solvent such as water or ethanol, by oxidation of the graphene planes with an HCl/CrO₃ mixture, described in the article “Room Temperature Filling of Single Wall Carbon Nanotubes With Oxide in open air,” J. Mittal et al. (Chem. Phys. Lett. 2001, vol. 339, No. 5-6, pages 311-318) or by condensation of an amino acid and an aldehyde onto the nanotubes (J. Am. Soc., vol. 124, No. 5, 2002, pages 760 and 761).

Hydrophobic functions may also be grafted onto the surface of the graphene planes constituting the carbon nanotube. Mention may be made, for example, of the fluorination of carbon nanotubes described in the article “Fluorinated Single Wall nanotubes,” K. N. Kudin et al. (Phys. Rev. B63, 45413).

Mineral molecules such as alkoxysilanes may also be grafted (Nano. Lett., vol. 2, No. 4, 2002 pages 329 to 332).

Functionalization of the graphene planes may be performed in several steps, for example, functionalization of the carbon nanotubes with fatty-chain amides, described in the article “Dissolution of Single Wall Carbon Nanotube,” M. A. Hamon et al. (Adv. Mater. 1999, 11, No. 10). This multi-step functionalization may also be used for the grafting of glucose (Nano. Lett., vol. 2, No. 4, 2002 pages 369-73).

Functionalization of the carbon nanotubes may be performed with simple molecules, but also with oligomers, polymers, and dendrimers. The article “A New Purification Method for Single Wall Carbon Nanotubes,” M. Holzinger et al. (Appl. Phys. A 70 (2000) 599) describes the grafting of dendritic structures onto the surface of graphene planes, constituting the carbon nanotube.

In addition to improving the dispersion of the carbon nanotubes in cosmetic media, the surface may also be functionalized to increase the affinity of the carbon-based nanostructures for the keratin material. The improvement in the affinity between the nanotubes and the keratin material induced by the functionalization of the graphene planes may be a result of increasing the Van der Waals interactions and/or a result of the creation of hydrogen bonds and/or ionic bonds. Thus, the functional group or groups are capable of creating with the keratin fibers, one or more chemical bonds chosen from Van der Waals interactions, hydrogen bonds, ionic bonds, and covalent bonds. The grafting of cationic molecules onto the surface of carbon nanotubes is described in the article “Exohedral Sidewall Reactions of Single Walled Carbon Nanotubes in Molecular Nanostructures,” M. Holzinger et al. (Proceeding of the XIIth International Winterschool on Electronic Properties of Novel Materials: Molecular Nanostructures, Kirchberg, Austria, March 2001). Cationic molecules may be grafted; the grafting of polyethyleneimine derivatives is described in Nano. Lett., vol. 2, No. 3, 2002 pages 231-34.

The nanotubes may also be polymeric.

The polymers used to obtain the nanoobjects are synthetic polymers. As used herein, the term “synthetic polymer” means a polymer obtained by chemical or electrochemical synthesis (free-radical polymerization, polycondensation, polymerization by ring opening, or polymerization by methathesis).

The crosslinking may take place chemically or under the action of photochemical radiation, for instance, under the action of UV or temperature. The polymers may be homopolymers or copolymers.

Homopolymers and copolymers derived from the free-radical polymerization of monomers comprising ethylenic, vinyl, allylic, (meth)acrylate and/or (meth)acrylamide units and derivatives may be used in at least one embodiment, such as vinyl/(meth)acrylate, vinyl/(meth)acrylamide, vinyl/(meth)acrylate/(meth)acrylamide, olefinic/vinyl copolymers, and (meth)acrylates/(meth)acrylamides. Polymer nanotubes are described in “Nanotube formation from renewable resources via coiled nanofibers,” G. John, M. Masuda, Y. Okada, K. Yase, T. Shimizu, Advanced Materials, 2001, 13, 715-718 and “Bottom-up synthesis and structural properties of self-assembled high-axial-ratio nanostructures,” T. Shimizu, Macromol. Rapid Commun., 2002, 23, 311-331.

The particles may also be chosen from semiconductive nanoparticles.

Luminescent semiconductive nanoparticles are capable of emitting, under the action of light excitation, radiation having a wavelength ranging from 400 to 700 nm in the cosmetics field

These nanoparticles have narrower color emission spectra than most dyes and organic pigments used in hair dyeing. Purer colorations are thus obtained, making it possible to obtain long-lasting optical effects.

As used herein, the term “optical effect” encompasses sheen, color, metallic, goniochromatic, and moiré effects.

The sheen corresponds to the light intensity reflected at an angle α when the lock of hair is illuminated at an angle −α. The angle α conventionally used to measure this specular reflection, i.e., the sheen, is equal to 200. The sheen may be measured by using a glossmeter, as described, for example, in standard ISO 2813-1994 from AFNOR (August 1994, amended in February 1997).

The color emitted by the nanoparticles varies as a function of their diameter. Thus, varied ranges of colors may be obtained by using one or more sizes of nanoparticles in the compositions. These nanoparticles also have the feature of emitting very strong colors.

In some embodiments, the nanoparticles comprise at least one metal chosen from Zn, Cd, and Hg and at least one metal chosen from S, Se, and Te, for example, cadmium selenide and cadmium sulfide.

The metals present in the nanoparticles may be uniformly distributed. Alternatively, the nanoparticles may also comprise a core comprising one or more metals and one or more layers covering the core, comprising one or more metals different from those constituting the core. These nanoparticles are known in the literature as core/shell nanoparticles.

In one embodiment, the nanoparticles have a cadmium selenide core covered with a layer of zinc sulfide.

The nanoparticles may also be covered with one or more additional organic and/or mineral layers, for example, having affinity for the hair. Examples of organic layers include layers obtained from polyethylene glycol, polyurethane, dextran, polyacrylic, polyvinylpyrrolidone, and polyvinylcaprolactone.

Examples of mineral layers include layers obtained from alumina, silica, clay, and mixtures thereof.

The layers may be obtained via a sol-gel process starting with organosilane. The layers, obtained by encapsulation of the nanoparticles, may be made via various processes, such as controlled precipitation, phase separation, emulsion polymerization, interfacial polycondensation, and in situ polycondensation.

Such encapsulation processes are described in “Microencapsulation Methods and Industrial Applications” (ISBN 0-8247-9703-5).

The capsules may be formed from any mineral compound, such as, a metal oxide or an organometallic polymer, for example, a metal oxide or an organometallic polymer obtained via a sol-gel process, such as the metal oxides or the organometallic polymers synthesized by polycondensation of one or a mixture of simple or mixed oxides of silicon, aluminium, boron, lithium, magnesium, sodium, titanium and/or zirconium. The nature of the precursors and the reaction mechanisms are described in “Sol Gel Science” by C. J Brinker and G. W. Scherer, published by Academic Press (ISBN 0-12-134970-5).

The additional layers may be covalently grafted or may be adsorbed onto the surface of the nanoparticles.

In other embodiments, the nanoparticles may be incorporated into polymer microbeads, wherein the polymer is chosen from hydrophilic, hydrophobic, amphiphilic, ionic, and nonionic polymers. The polymers include the polystyrenes described in: “Quantum-dot-tagged microbeads for multiplexed optical coding of biomolecules,” Mingyong Han, Nature Biotechnology Vol. 19, pp. 631-635 July 2001.

The nanoparticles may have sizes ranging from 1 to 100 nm, for example, from 1 to 50 nm or from 1 to 20 nm.

Nanoparticles are known in the literature and may be manufactured according to the processes described, for example, in U.S. Pat. Nos. 6,225,198 and 5,990,479 and in the publications cited therein, and also in the following publications:

Dabboussi B. O. et al “(CdSe)ZnS core-shell quantum dots: synthesis and characterisation of a size series of highly luminescent nanocrystallites” Journal of Physical Chemistry B, vol. 101, 1997, pp. 9463-9475 and

Peng, Xiaogang et al., “Epitaxial Growth of highly Luminescent CdSe/CdS core/shell nanocrystals with photostability and electronic accessibility” Journal of the American Chemical Society, vol. 119, No. 30, pp. 7019-7029. Nanoparticles known as “quantum dots” may also be used.

Examples include the following nanoparticles:

| Type of | | | Solution |
|---|---|---|---|
| nanoparticles | Size | Color | concentration |
| CdSe | 2.2 nm | Green | 0.5 mg/ml |
| CdSe | 3.4 nm | Yellow | 0.5 mg/ml |
| CdSe | 4.0 nm | Orange | 0.5 mg/ml |
| CdSe | 4.7 nm | Orange-red | 0.5 mg/ml |
| CdSe | 5.6 nm | Red | 0.5 mg/ml |
| CdSe/ZnS | 4.3 nm | Green | 0.5 mg/ml |
| CdSe/ZnS | 4.8 nm | Green-yellow | 0.5 mg/ml |
| CdSe/ZnS | 5.4 nm | Yellow | 0.5 mg/ml |
| CdSe/ZnS | 6.3 nm | Orange | 0.5 mg/ml |
| CdSe/ZnS | 7.2 nm | Red | 0.5 mg/ml |

These nanoparticles are sold by the company Evident Technologies.

CdSe nanoparticles are uniform nanoparticles that contain only CdSe. CdSe/ZnS nanoparticles have core/shell structures with a CdSe core and a ZnS shell.

Nanofibrils or microfibrils may be used as nanoparticles or microparticles. As used herein, the term “nanofibrils or microfibrils” means particles as described in the following publications:
Polymerization in nanometer-sized fibers: Molecular Packing Order and Polymerizability: M. Masuda, T. Hanada, Y. Okada, K. Yase, T. Shimizu, Macromolecules, 2000, 33, 9233-9238, and
Organic supramolecular self-assembled materials stabilized by multiple hydrogen bonds, T. Shimizu, Transactions of the Materials Research Society of Japan, 1999, 24, 3, 431-436.

The fibrils may be natural, such as cellulose, proteins or silk, or synthetic, such as polyamide.

The microparticles or nanoparticles may also be chosen from expandable particles, for example, compounds capable of swelling under the action of heat.

Some expandable particles, may react, under the action of heat, to liberate a gas that is trapped in the matrix of the deposit.

Compounds capable of swelling under the action of heat include heat-expandable particles.

As used herein, the term “heat-expandable particles” means particles capable of becoming deformed and of expanding with heat. The particles may also be heat-deformable non-expanded particles. These particles are distinguished in this respect from expanded particles, because they are no longer subject to deformation under the action of heat, such as, for example, polyvinylidene/acrylonitrile particles sold under the general name “Expancel®” by the company Akzo Nobel under the names “Expancel® WE” and “Expancel® DE.”

These particles are capable of expanding under the action of a temperature generally of greater than or equal to 45° C., for example, greater than or equal to 50° C., greater than or equal to 60° C., greater than or equal to 70° C., greater than or equal to 80° C., greater than or equal to 85° C., greater than or equal to 90° C., and ranging up to from 190 to 200° C.

In some embodiments, the particles are not sensitive to the presence of water.

In some embodiments, the particles are thermoplastic. As used herein, the term “thermoplastic” means particles that are capable of becoming deformed under the action of heat and of keeping their new shape, including after cooling to room temperature.

In some embodiments, the particles are generally hollow particles comprising a continuous envelope and at least one cavity.

In some embodiments, the envelope of the particles is flexible so as to lend itself to mechanical deformation. It generally comprises at least one polymer that is a homopolymer or copolymer formed from ethylenically unsaturated monomers. Examples of such particles are described in EP-A-56219, EP-A-348 372, EP-A-486 080, EP-A-320 473, EP-A-1 12 807 and U.S. Pat. No. 3,615,972.

The monomers used may be chosen from methacrylic and acrylic acid esters, such as methyl acrylate and methacrylate, vinylidene chloride, acrylonitrile, styrene and its derivatives, butadiene and its derivatives, and mixtures thereof.

Polymers that may be used to comprise the envelope of the particles include, for example, polymers comprising at least some units derived from methyl acrylate or methacrylate, polymers comprising at least some units derived from acrylonitrile, polymers comprising at least some units derived from acrylonitrile and from methyl methacrylate, polymers comprising at least some units derived from styrene and from acrylonitrile, polymers comprising at least some units derived from vinylidene chloride and from acrylonitrile, and polymers comprising at least some units derived from vinylidene chloride and from vinyl chloride. In some embodiments, the polymer may be chosen from vinylidene chloride/acrylonitrile/methyl methacrylate polymers, acrylonitrile/methyl methacrylate polymers, and acrylonitrile homopolymers.

The particles may comprise within one or more cavities at least one compound capable of showing a significant increase in its volume at room temperature in response to heating to a temperature ranging from 45 to 200° C. and at a substantially constant pressure.

As used herein, the term “significant increase in its volume” means an increase by at least a factor of 30, for example, by at least a factor of 40 or 50, of the occupied volume.

In general, the compound contained within the cavity may be, at room temperature, a gaseous compound or a liquid compound having a vaporization temperature ranging from 45 to 200° C., for example, from 80 to 200° C. or greater than or equal to 100° C.

In some embodiments, the compound is in gaseous form in the particle and dilates under the effect of heat. Compounds in gaseous form include air, nitrogen, hydrocarbons containing 1, 2, 3 or 4 carbon atoms such as butane or isobutane, and mixtures thereof.

In other embodiments, the compound in the cavity is a liquid compound as defined above. These compounds include hydrocarbons, for example, having from 5 to 15 carbon atoms, such as from 5 to 12 or from 5 to 10 carbon atoms. The compound in the cavity may be a compound chosen from n-pentane, isopentane, and neopentane.

The expansion temperature of the particle depends both on the nature of the compound present in its cavity and on that of the polymer forming its envelope, and may range from 45 to 200° C., for example, and may be greater than or equal to 80° C. or greater than or equal to 100° C.

The particles used in the compositions disclosed herein may be dry or hydrated.

These particles may be in various forms. For example, they may have a globular or spherical form, or may be elongated.

In some embodiments, the non-expanded particles disclosed herein are spherical and have a particle size, expressed as the weight-average “effective” diameter, D[0.5], ranging from 0.5 to 200 μm, for example, from 1 to 100 μm, from 4 to 50 μm, and from 5 to 40 μm.

In some embodiments, the particles used in the compositions have a fiber shape. As used herein, the term “fiber” means an object of length L and diameter D such that L is greater than D, D being the diameter of the circle within which the cross section of the fiber is inscribed. The ratio L/D (or shape factor) is chosen within the range from 3.5 to 2500, for example, from 5 to 500 or from 5 to 150. In some embodiments, the fibers have a length ranging from 0.05 to 6 mm.

The non-expanded particles may have a mass per unit volume ranging from 500 to 5000 kg/m³, for example, from 900 to 3000 kg/m³ or from 900 to 2000 kg/m³.

Particles that may be used in the compositions of the present disclosure include non-expanded microspheres of vinylidene chloride/acrylonitrile/methyl methacrylate copolymer, for example, those sold under the name “Expancel®” by the company Akzo Nobel under the references 820DU 40 (10-16 μm) and 820 SL 40 (2-30 μm), and of acrylonitrile/methyl methacrylate copolymer, for instance those sold under the name “Expancel®” under the references 642 WU 40 (10-16 μm) and 051 DU 40 (9-15 μm). Particles that may be used in the compositions also include non-expanded microspheres of acrylonitrile homopolymer, for example, those sold under the name “Expancel 007W®” (5-25 μm), “Micropearl F-series®” by the company Matsumoto, and “Ucelite®” by the company UCB.

The particles sold under the name “Expancel®” under the references listed above generally comprise at least one compound in gaseous form in their cavities.

The microparticles or nanoparticles may be present in the compositions in an amount ranging from 0.0001 to 30% by weight, for example, from 0.001% to 20% or from 0.01 to 10% by weight, relative to the total weight of the composition.

As used herein, the term “electrophilic monomer” means a monomer capable of polymerizing via anionic polymerization in the presence of a nucleophilic agent, for example, hydroxyl ions (—OH) in water.

As used herein, the term “anionic polymerization” is the mechanism as defined in the book “Advanced Organic Chemistry,” Third Edition, by Jerry March, pages 151 to 161.

Electrophilic monomers present in the compositions may be chosen from:

benzylidene malonitrile derivatives (A), 2-(4-chloro-benzylidene)malononitrile (A1), ethyl 2-cyano-3-phenylacrylate (B), and ethyl 2-cyano-3-(4-chlorophenyl)acrylate (B1) described in Sayyah, J. Polymer Research, 2000, p. 97:

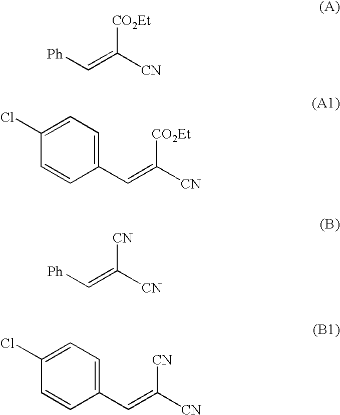

methylidenemalonate derivatives, for example:
diethyl 2-methylenemalonate (C) described in Hopff, Makromoleculare Chemie, 1961, p. 95, De Keyser, J. Pharm. Sci, 1991, p. 67 and Klemarczyk, Polymer, 1998, p. 173: 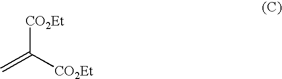
ethyl 2-ethoxycarbonylmethylenecarbonylacrylate (D) described in Breton, Biomaterials, 1998, p. 271 and Couvreur, Pharmaceutical Research, 1994, p. 1270:

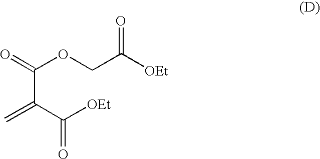

itaconate and itaconimide derivatives, for example:
dimethyl itaconate (E) described in Bachrach, European Polymer Journal, 1976, p. 563: 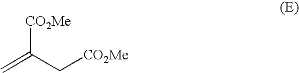
N-butyl itaconimide (F), N-(4-tolyl) itaconimide (G), N-(2-ethylphenyl) itaconimide (H), N-(2,6-diethylphenyl) itaconimide (I) described in Wanatabe, J. Polymer Science: Part A: Polymer chemistry, 1994, p. 2073: 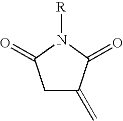
   R═Bu (F), 4-tolyl (G), 2-ethylphenyl (H), 2,6-diethylphenyl (I) the derivatives methyl α-(methylsulfonyl)acrylate (K), ethyl α-(methylsulfonyl)acrylate (L), methyl α-(tert-butylsulfonyl)acrylate (M), tert-butyl α-(methylsulfonyl)acrylate (N) and tert-butyl α-(tert-butylsulfonyl)acrylate (O), described in Gipstein, J. Org. Chem, 1980, p. 1486; and

the derivatives 1,1-bis(methylsulfonyl)ethylene (P), 1-acetyl-1-methylsulfonylethylene (O), methyl α-(methylsulfonyl)vinylsulfonate (R) and α-methylsulfonylacrylonitrile (S) by Shearer, described in U.S. Pat. No. 2,748,050:

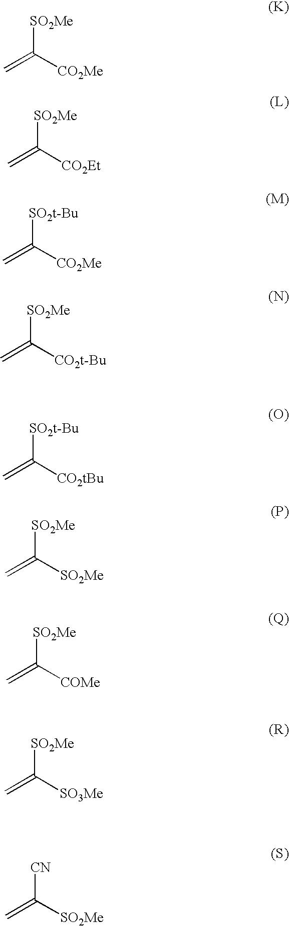

methyl vinyl sulfone (T) and phenyl vinyl sulfone (U) derivatives described in Boor, J. Polymer Science, 1971, p. 249:

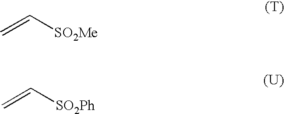

the phenyl vinyl sulfoxide derivative (V) described in Kanga, Polymer preprints (ACS, Division of Polymer Chemistry), 1987, p. 322:

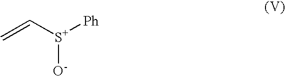

the derivative 3-methyl-N-(phenylsulfonyl)-1-aza-1,3-butadiene (W) described in Bonner, Polymer Bulletin, 1992, p. 517:

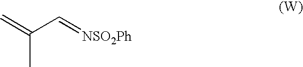

acrylate and acrylamide derivatives, for example:

N-propyl-N-(3-triisopropoxysilylpropyl)acrylamide (X) and N-propyl-N-(3-triethoxysilylpropyl)acrylamide (Y) described in Kobayashi, Journal of Polymer Science, Part A: Polymer Chemistry, 2005, p. 2754:

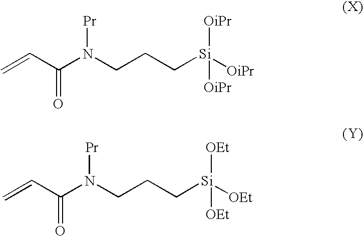

2-hydroxyethyl acrylate (Z) and 2-hydroxyethyl methacrylate (AA) described in Rozenberg, International Journal of Plastics Technology, 2003, p. 17:

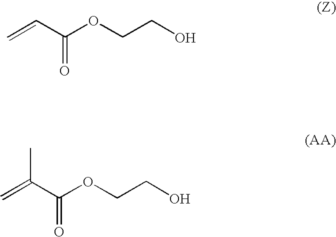

N-butyl acrylate (AB) described in Schmitt, Macromolecules, 2001, p. 2115 and tert-butyl acrylate (AC) by Ishizone, Macromolecules, 1999, p. 955:

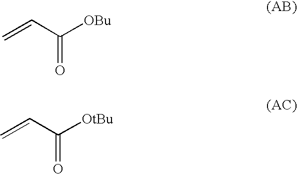

The electron-withdrawing monomers that may be used may be cyclic or linear. When cyclic, the electron-withdrawing groups may be exocyclic, i.e., do not form an integral part of the cyclic structure of the monomer.

In some embodiments, the monomers comprise at least two electron-withdrawing groups.

Examples of monomers comprising at least two electron-withdrawing groups include monomers of formula (I): 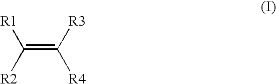
wherein:
R¹ and R², independently of each other, are each chosen from a sparingly or non-electron-withdrawing group (sparingly or non-inductive-withdrawing) such as:
a hydrogen atom,
saturated or unsaturated, linear, branched or cyclic hydrocarbon-based groups comprising from 1 to 20 (e.g., from 1 to 10) carbon atoms, optionally comprising one or more atoms chosen from nitrogen, oxygen and sulfur atoms, and optionally substituted with one or more groups chosen from —OR, —COOR, —COR, —SH, —SR and —OH, and halogen atoms,
modified or unmodified polyorganosiloxane residues,
polyoxyalkylene groups,
R³ and R⁴, independently of each other, are each chosen from an electron-withdrawing (or inductive-withdrawing) group, for example, chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups such as phenyl, and aryloxy groups such as phenoxyloxy,

R is chosen from a saturated or unsaturated, linear, branched or cyclic hydrocarbon-based group comprising from 1 to 20 (e.g., from 1 to 10) carbon atoms, and optionally comprising one or more atoms chosen from nitrogen, oxygen and sulfur atoms, and optionally substituted with one or more groups chosen from —OR′, —COOR′, —COR′, —SH, —SR′ and —OH, halogen atoms, or a polymer residue, wherein R′ denotes a C₁-C₁₀ alkyl radical, optionally obtained by free-radical polymerization, by polycondensation or by ring opening.

As used herein, the term “electron-withdrawing or inductive-withdrawing group (—I)” means any group that is more electronegative than carbon. Electron-withdrawing or inductive-withdrawing group are described in P. R. Wells, Prog. Phys. Org. Chem., Vol 6111 (1968).

As used herein, the term “sparingly or non-electron-withdrawing group” means any group having an electronegativity less than or equal to that of carbon.

The alkenyl and alkynyl groups may have from 2 to 20 carbon atoms, for example, from 2 to 10 carbon atoms.

Saturated or unsaturated, linear, branched or cyclic hydrocarbon-based groups may have from 1 to 20 carbon atoms (e.g., from 1 to 10 carbon atoms), for example, linear or branched alkyl, alkenyl or alkynyl groups, such as methyl, ethyl, n-butyl, tert-butyl, isobutyl, pentyl, hexyl, octyl, butenyl, and butynyl; cycloalkyl; and aromatic groups.

Examples of substituted hydrocarbon-based groups include hydroxyalkyl and polyhaloalkyl groups.

Examples of unmodified polyorganosiloxanes include polyalkylsiloxanes such as polydimethylsiloxanes, polyarylsiloxanes such as polyphenylsiloxanes, and polyarylalkylsiloxanes such as polymethylphenylsiloxanes.

Examples of modified polyorganosiloxanes include polydimethylsiloxanes comprising polyoxyalkylene and/or siloxy and/or silanol and/or amine and/or imine and/or fluoroalkyl groups.

Examples of polyoxyalkylene groups include polyoxyethylene groups and polyoxypropylene groups, for example, having from 1 to 200 oxyalkylene units.

Mono- and polyfluoroalkyl groups include —(CH₂)ₙ—(CF₂)ₙ—CF₃ and —(CH₂)ₙ—(CF₂)ₘ—CHF₂ wherein n ranges from 1 to 20 and m ranges from 1 to 20.

The substituents R₁ to R₄ may optionally be substituted with a group having cosmetic activity. Cosmetic activities that may be used are obtained from groups having coloring, antioxidant, UV-screening, and conditioning functions.

Examples of groups having a coloring function include azo, quinone, methine, cyanomethine, and triarylmethane groups.

Examples of groups having ananntioxidant function include butylhydroxyanisole (BHA), butylhydroxytoluene (BHT), and vitamin E groups.

Examples of groups having a UV-screening function include benzophenone, cinnamate, benzoate, benzylidenecamphor, and dibenzoylmethane groups.

Examples of groups having a conditioning function include cationic groups and fatty ester groups.

In some embodiments, the monomers are monomers of the cyanoacrylate family and derivatives thereof of formula (II): 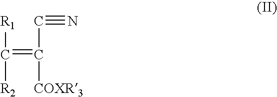
wherein:
   X is chosen from NH, S, and O,
   R₁ and R₂ are as described above,
   R₁₃ is chosen from a hydrogen atom and a radical R as defined for formula (I).

In some embodiments, X is O.

Compounds of formula (II) that may be used include the following monomers:

(a) C₁-C₂₀ polyfluoroalkyl 2-cyanoacrylates, such as, the ester 2,2,3,3-tetrafluoropropyl 2-cyano-2-propenoate of formula: 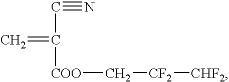
or the ester 2,2,2-trifluoroethyl 2-cyano-2-propenoate of formula:

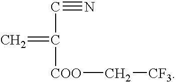

(b) the C₁-C₁₀ alkyl or (C₁-C₄ alkoxy)(C₁-C₁₀ alkyl) cyanoacrylates.

These monomers include ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

In some embodiments, the monomers (b) listed above are used.

In some embodiments, monomers of formula II and mixtures thereof are used: 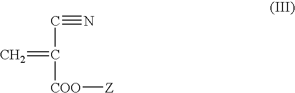
wherein:
Z is chosen from —(CH₂)₇—CH₃,
   —CH(CH₃)—(CH₂)₅—CH₃,
   —CH₂—CH(C₂H₅)—(CH₂)₃—CH₃,
   —(CH₂)₅—CH(CH₃)—CH₃, and
   —(CH₂)₄—CH(C₂H₅)—CH₃.

The monomers may be covalently bonded to supports such as polymers, oligomers, and dendrimers. The polymers or oligomers may be linear, branched, in comb form, or in block form. The distribution of the monomers in the polymeric, oligomeric or dendritic structure may be random, in terminal positions, or in the form of blocks.

As used herein, the term “cosmetically acceptable medium” means a medium that is compatible with keratin materials such as the hair.

The cosmetically acceptable medium may be anhydrous. As used herein, the term “anhydrous medium” means a medium containing less than 1% by weight of water relative to the total weight of the composition.

The cosmetically acceptable medium may be chosen from organic oils; silicones such as volatile silicones, amino and non-amino silicone gums and oils and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil, and lemon oil; waxes; and organic compounds such as C₅-C₁₀ alkanes, acetone, methyl ethyl ketone, esters of C₁-C₂₀ acids and of C₁-C₈ alcohols such as methyl acetate, butyl acetate, ethyl acetate and isopropyl myristate, dimethoxyethane, diethoxyethane, C₁₀-C₃₀ fatty alcohols such as lauryl alcohol, cetyl alcohol, stearyl alcohol and behenyl alcohol; C₁₀-C₃₀ fatty acids such as lauric acid and stearic acid; C₁₀-C₃₀ fatty amides such as lauric diethanolamide, and C₁₀-C₃₀ fatty alkyl esters such as C₁₀-C₃₀ fatty alkyl benzoates, and mixtures thereof.

In some embodiments, the organic compounds are chosen from compounds that are liquid at a temperature of 25° C. and at 105 Pa (760 mm Hg).

The compositions may have a concentration of electrophilic monomers ranging from 0.001 to 80% by weight, such as from 0.1 to 40% or from 1% to 20% by weight, relative to the total weight of the composition.

Polymerization inhibitors such as anionic and/or free-radical polymerization inhibitors may also be introduced into the compositions to enhance the stability of the composition over time. Polymerization inhibitors include: sulfur dioxide; nitric oxide; lactone; boron trifluoride; hydroquinone and derivatives thereof such as hydroquinone monoethyl ether; tert-butylhydroquinone (TBHQ); benzoquinone and derivatives thereof such as duroquinone; catechol and derivatives thereof such as t-butylcatechol and methoxycatechol; anisole and derivatives thereof such as methoxyanisole, hydroxyanisole and butylhydroxyanisole; pyrogallol; 2,4-dinitrophenol; 2,4,6-trihydroxybenzene; p-methoxyphenol; hydroxybutyltoluene; alkyl sulfates; alkyl sulfites; alkyl sulfones; alkyl sulfoxides; alkyl sulfides; mercaptans; 3-sulfonene; and mixtures thereof. In some embodiments, the alkyl groups have from 1 to 6 carbon atoms.

It is also possible to use mineral or organic acids, the latter containing one or more carboxylic or sulfonic groups, with a pKa ranging from 0 to 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid, and trifluoroacetic acid.

The amount of inhibitor may range from 10 ppm to 20%, such as from 10 ppm to 5% or 10 ppm to 1%, by weight relative to the total weight of the composition.

The compositions may further comprise at least one agent known to be used in cosmetics, for example, reducing agents, fatty substances, plasticizers, softeners, antifoams, moisturizers, pigments, UV-screening agents, peptizers, solubilizing agents, fragrances, preserving agents, anionic surfactants, cationic surfactants, nonionic surfactants, amphoteric surfactants, fixing or non-fixing polymers, polyols, proteins, vitamins, direct dyes or oxidation dyes, nacreous agents, mineral or organic thickeners such as benzylidene sorbitol, and N-acylamino acids. These agents may optionally be encapsulated, for example, in a polycyanoacrylate capsule.

The treatment processes for keratin fibers disclosed herein comprise applying the compositions described above to keratin materials, and optionally in the presence of a nucleophilic agent with or without heating.

The nucleophilic agent may be water and may be provided by wetting the keratin material beforehand.

To modify the reaction kinetics, the keratin materials may be wetted beforehand using an aqueous solution whose pH has been adjusted using a base, an acid or an acid/base mixture. The acid and/or the base may be mineral or organic.

These two operations may also be performed after applying the composition.

It is also possible to modify the anionic polymerization kinetics by preimpregnating the keratin materials with a nucleophilic agent. The nucleophilic agent may be used pure, as a solution, in the form of an emulsion, or may be encapsulated.

The nucleophilic agents capable of initiating the anionic polymerization are capable of generating a carbanion on contact with a nucleophilic agent, such as the hydroxyl ions contained in water. As used herein, the term “carbanion” means the chemical species defined in “Advanced Organic Chemistry, Third Edition,” by Jerry March, page 141.

The nucleophilic agents may comprise a molecular compound, an oligomer, a dendrimer, or a polymer containing nucleophilic functions. Nucleophilic functions include the following functions: ⁻NR₂, NH₂⁻, Ph₃C⁻, R₃C⁻, PhNH⁻, pyridine, ArS⁻, R—C≡C⁻, RS⁻, ⁻SH, RO⁻, R₂NH, ArO⁻, N₃⁻, ⁻OH, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group; Ar is an aryl group, and R is a C₁-C₁₀ alkyl group.

In some embodiments, the nucleophilic agent is water. The water may be provided by moistening beforehand.

To modify the reaction kinetics, the fiber may be moistened beforehand using an aqueous solution whose pH has been adjusted using a base, an acid or an acid/base mixture. The acid and/or the base may be mineral or organic.

It is also possible to modify the anionic polymerization kinetics by preimpregnating the fiber with a nucleophilic agent other than water. The nucleophilic agent may be used pure, as a solution, in the form of an emulsion or may be encapsulated. To modify the anionic polymerization kinetics, it is also possible to increase the nucleophilicity of the fiber via chemical conversion of the keratin material. One such chemical conversion is the reduction of the disulfide bridges of which keratin is partly composed, into thiols, before applying the composition. Suitable reducing agents for the disulfide bridges of which keratin is partially composed include anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, and monoethanolamine thioglycolate.

The viscosity of the compositions can be increased to modify the anionic polymerization kinetics, for example, to reduce the rate of polymerization of the monomers. To do this, one or more polymers that have no reactivity towards the monomers disclosed herein may be added to the composition of the present disclosures. Suitable polymers include poly(methyl methacrylate) (PMMA) and cyanoacrylate-based copolymers as described in U.S. Pat. No. 6,224,622.

To improve, inter alia, the adhesion of the poly(cyanoacrylate) formed in situ, the fibers may be pretreated with polymers of any type, or a hair treatment may be performed before applying the compositions, for instance a direct dyeing or oxidation dyeing, permanent-waving, or hair relaxing operation.

The disclosure also provides novel compositions as defined above. In these compositions, the microparticles or nanoparticles may be mineral, organic or mixed, and may be coated or grafted. For example, the particles may be metal oxides, polymer particles, quantum dots, nanotubes, or nanofibrils. The compositions may be as described above.

The application of the compositions may or may not be followed by rinsing. These compositions may be in various forms, such as in the form of lotions, sprays or mousses, and may be applied in the form of a shampoo or a hair conditioner.

The mode of application may be in a single step or alternatively may be divided into successive steps. When the process includes several steps of application of active compositions, the steps may be as follows:
(1) application to the hair of the microparticles or nanoparticles, present in an aqueous solution in a proportion ranging from 0.05 to 40%, for example, from 0.1 to 35% or from 0.25% to 25%,
(2) application of the monomer to moistened hair, wherein the monomer is present in solution in a concentration ranging from 0.05 to 30% by weight, for example, from 0.01 to 50% by weight or from 0.1 to 20% by weight.

The order of steps (1) and (2) may be inverted. The first step may be preceded by the application of a cosmetic product. Similarly, the second step may be succeeded by the application of a cosmetic product. Each step may be interrupted by rinsing or drying. The drying may be performed under a drying hood, with a hairdryer and/or with a smoothing iron.

In addition to the active agent, each composition may also further comprise conventional cosmetic additives.

The monomers may be chosen from monomers capable of polymerizing on the keratin fibers under cosmetically acceptable conditions. The polymerization of the monomer may be performed at a temperature of less than or equal to 80° C., for example, from 10 to 80° C. or 20 to 80° C., which does not prevent the application from being completed by drying under a drying hood, blow-drying or treating with a flat iron or a crimping iron.

The disclosure also provides uses of the compositions described above for the cosmetic treatment of keratin materials, including keratin fibers such as the hair. The compositions may be used for reinforcing keratin materials, keratin fibers, for example, reinforcing the hair or nails.

The process may include a step of applying microparticles or nanoparticles to the keratin materials and a step of applying at least one electrophilic monomer to the keratin materials, or performing the steps in the opposite order.

In some embodiments, the application of the microparticles or nanoparticles is performed before the application of the electrophilic monomers.

The presently disclosed invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLES

Tests were performed using the following compounds:
Monomer: n-octyl 2-cyanoacrylate, sold under the name “Rite Lok CON895” by the company Chemence;
Particles 1: polytetrafluoroethylene powder as an aqueous 50% dispersion (size: 900 nm) protected (parabens) sold under the name “Fluoropure Ultrafine 50CW” by Shamrock Technologies;
Particles 2: non-expanded enveloped microspheres: vinylidene chloride/acrylonitrile/methyl methacrylate (10-16 microns) sold under the name “Expancel 820 DU 40;”

Cosmetically acceptable medium: 50% mixture of alpha-omega dihydroxyl polydimethylsiloxane/cyclopentadimethylsiloxane (14.7/85.3) sold by Dow Corning under the name “DC 1501 Fluid,” with 50% cyclopentadimethylsiloxane sold by Dow Corning under the name “DC 245 Fluid.”

### Example 1

An aqueous dispersion was prepared with 10% of Particle Type 1. The aqueous solution (0.5 g) was applied to 1 g of a lock of clean and dry natural hair with a tone height of 4, which corresponds to a natural chestnut shade according to the classification of natural shades described in “The Science of Hair Care” by C. Zviak, published by Masson, 1988, p. 278.

The lock was dried under a drying hood, and then moistened with 0.5 g of water. To this moistened lock was applied 0.5 g of a composition comprising the cosmetically acceptable medium described above and 10% by weight of cyanoacrylate monomer.

After a leave-in time of 10 minutes, the lock was dried for 2 minutes with a hairdryer.

The lock obtained had a larger volume than both an untreated lock and a lock that was treated with the same composition but without the incorporation of particles.

### Example 2

An aqueous dispersion was prepared with 10% of Particle Type 1. The aqueous solution (0.5 g) was applied to 1 g of a lock of clean and dry natural hair with a tone height of 4, which corresponds to a natural chestnut shade. To this lock was then applied 0.5 g of a composition comprising the cosmetically acceptable medium described above and 10% by weight of cyanoacrylate monomer.

After a leave-in time of 10 minutes, the lock was dried for 2 minutes with a hairdryer.

The lock obtained had a larger volume than both an untreated lock and a lock that was treated with the same composition but without the incorporation of particles.

### Example 3

A composition was prepared containing 1% of Particle Type 2 in the cosmetically acceptable medium described above. A cyanoacrylate monomer was added to the composition so as to obtain a final concentration of 10% by weight of monomer. The composition (0.5 g) was applied to a lock of clean and dry natural hair with a tone height equal to 4, moistened with 0.5 g of water.

After a leave-in time of 10 minutes, the lock was dried for 2 minutes with a hairdryer.

The lock obtained had a larger volume than both an untreated lock and a lock that was treated with the same composition but without the incorporation of particles.

In Examples 4 to 10, non-expanded microspheres of vinylidene chloride/acrylonitrile/methyl methacrylate copolymer (10-16 microns) sold under the name “Expancel 820 DU 40” by the company Akzo Nobel (Particle Type 2) were used.

### Example 4

The following composition was prepared:

| | |
|---|---|
| DC 1501 Fluid | 45 g |
| DC 245 Fluid | 42 g |
| Expancel 820 DU 40 | 3 g |
| Methylheptyl cyanoacrylate from Chemence | 10 g |

The composition (0.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of methylheptyl cyanoacrylate.

### Example 5

The following composition was prepared:

| | | |
|---|---|---|
| DC 1501 Fluid | 45 | g |
| DC 245 Fluid | 41.75 | g |
| Expancel 820 DU 40 | 3 | g |
| Acetic acid | 0.25 | g |
| Methylheptyl cyanoacrylate from Chemence | 10 | g |

The composition (0.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of methylheptyl cyanoacrylate.

### Example 6

The following composition was prepared:

| | |
|---|---|
| DC 1501 Fluid | 45 g |
| DC 245 Fluid | 37 g |
| Expancel 820 DU 40 | 3 g |
| Ethoxyethyl cyanoacrylate EO-460 from Tong Shen | 10 g |
| Acetic acid | 5 g |

The composition (1.5 g) was applied to a lock of 1 g of clean wet hair. After a leave-in time of 15 minutes, the lock was dried with a hairdryer for ˜2 minutes.

The lock obtained had a larger volume than both an untreated lock and a lock that was treated with the same composition but without the incorporation of ethoxyethyl cyanoacrylate.

### Example 7

The following composition was prepared:

| | |
|---|---|
| DC 1501 Fluid | 45 g |
| DC 245 Fluid | 41 g |
| Expancel 820 DU 40 | 3 g |
| Butyl cyanoacrylate B-60 from Tong Shen | 10 g |
| Acetic acid | 1 g |

The composition (1.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of butyl cyanoacrylate.

### Example 8

The following composition was prepared:

| | |
|---|---|
| DC 1501 Fluid | 45 g |
| DC 245 Fluid | 42 g |
| Expancel 820 DU 40 | 3 g |
| Ethylhexyl cyanoacrylate O-60 from Tong Shen | 10 g |

The composition (0.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of ethylhexyl cyanoacrylate.

### Example 9

The following composition was prepared:

| | |
|---|---|
| DC 1501 Fluid | 45 g |
| DC 245 Fluid | 44 g |
| Expancel 820 DU 40 | 1 g |
| Methylheptyl cyanoacrylate from Chemence | 9 g |
| Ethylhexyl cyanoacrylate O-60 from Tong Shen | 1 g |

The composition (0.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of methylheptyl cyanoacrylate or ethylhexyl cyanoacrylate.

### Example 10

The following composition was prepared:

| | |
|---|---|
| DC 1501 Fluid | 45 g |
| DC 245 Fluid | 44 g |
| Expancel 820 DU 40 | 1 g |
| Methylheptyl cyanoacrylate from Chemence | 7 g |
| Butyl cyanoacrylate B-60 from Tong Shen | 3 g |

The composition (0.5 g) was applied to a lock of 1 g of clean wet hair. After a leave-in time of 15 minutes, the lock was dried with a hairdryer for 2 minutes.

The lock obtained had a larger volume than both an untreated lock and a lock that was treated with the same composition but without the incorporation of methylheptyl cyanoacrylate and butyl cyanoacrylate.

### Example 11

The following composition was prepared:

| | |
|---|---|
| DC 1501 Fluid | 45 g |
| DC 245 Fluid | 42 g |
| Talc E0326 sold by the company Luzenac | 3 g |
| Methylheptyl cyanoacrylate from Chemence | 10 g |

The composition (0.5 g) was applied to a lock of 1 g of clean wet hair. After a leave-in time of 15 minutes, the lock was dried with a hairdryer for 2 minutes.

A lock having a coat that withstood several shampoo washes was obtained.

### Example 12

The following composition was prepared:

| | |
|---|---|
| DC 1501 Fluid | 45 g |
| DC 245 Fluid | 42 g |
| Ultrafine Zinc Oxide - 350 | 3 g |
| (sold by the company Sumitomo) | |
| Methylheptyl cyanoacrylate from Chemence | 10 g |

The composition (0.5 g) was applied to a lock of 1 g of clean wet hair. After a leave-in time of 15 minutes, the lock was dried with a hairdryer for 2 minutes.

A lock having a coat that withstood several shampoo washes was obtained.

What is claimed is:

1. A method for treating keratin materials comprising applying to keratin materials a composition comprising, in a cosmetically acceptable medium, at least one electrophilic monomer and microparticles or nanoparticles.

2. The method of claim 1, wherein the keratin materials are hair.

3. The method according to claim 1, wherein the at least one electrophilic monomer is chosen from compounds of formula (I): 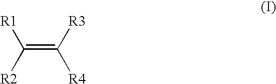
wherein:
R₁ and R₂ are independently chosen from a sparingly or non-electron-withdrawing group chosen from:
a hydrogen atom,
saturated or unsaturated, linear, branched or cyclic hydrocarbon-based groups, optionally comprising at least one atom chosen from nitrogen, oxygen, and sulfur atoms, and optionally substituted with at least one group chosen from —OR, —COOR, —COR, —SH, —SR and —OH, and halogen atoms,
modified and unmodified polyorganosiloxane residues, and
polyoxyalkylene groups; and
R₃ and R₄ are independently chosen from electron-withdrawing groups chosen from: —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear and branched alkenyl groups, linear and branched alkynyl groups, C₁-C₄ monofluoroalkyl and polyfluoroalkyl groups, and aryl and aryloxy groups,
R is chosen from saturated or unsaturated, linear, branched or cyclic hydrocarbon-based groups, optionally comprising at least one atom chosen from nitrogen, oxygen and sulfur atoms, and optionally substituted with at least one group chosen from —OR′, —COOR′, —COR′, —SH, —SR′ and —OH, halogen atoms, or a polymer residue, and
R′ is a C₁-C₁₀ alkyl radical.

4. The method of claim 3 wherein at least one of R₁, R₂, and R is chosen from saturated and unsaturated, linear, branched and cyclic hydrocarbon-based groups comprising from 1 to 20 carbon atoms.

5. The method according to claim 3, wherein the at least one electrophilic monomer is chosen from compounds of formula (II): 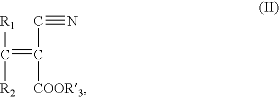
wherein
R₁ and R₂ are defined as in claim 3, and
R₁₃ is chosen from hydrogen and from saturated or unsaturated, linear, branched or cyclic hydrocarbon-based groups, optionally comprising at least one atom chosen from nitrogen, oxygen and sulfur atoms, and optionally substituted with at least one group chosen from —OR′, —COOR′, —COR′, —SH, —SR′ and —OH, halogen atoms, or a polymer residue, and R′ is a C₁-C₁₀ alkyl radical.

6. The method according to claim 5, wherein the at least one electrophilic monomer is chosen from C₁-C₂₀ polyfluoroalkyl 2-cyanoacrylates, (C₁-C₁₀) alkyl cyanoacrylates, and (C₁-C₄ alkoxy)(C₁-C₁₀ alkyl) cyanoacrylates.

7. The method according to claim 6, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

8. The method according to claim 5, wherein the at least one electrophilic monomer is chosen from compounds of formula (III): 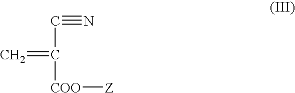
wherein Z is chosen from —(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

9. The method according to claim 1, wherein the at least one electrophilic monomer is present in the composition in an amount ranging from 0.001 to 80% by weight relative to the total weight of the composition.

10. The method according to claim 9, wherein the at least one electrophilic monomer is present in the composition in an amount ranging from 1 to 20% by weight relative to the total weight of the composition.

11. The method according to claim 1, wherein the at least one electrophilic monomer is covalently bonded to a support.

12. The method according to claim 11, wherein the support is chosen from polymers, oligomers, and dendrimers.

13. The method according to claim 1, wherein the cosmetically acceptable medium is anhydrous.

14. The method according to claim 1, wherein the cosmetically acceptable medium is chosen from organic oils, silicones, mineral oils, plant oils, waxes, C₅-C₁₀ alkanes, acetone, methyl ethyl ketone, esters of C₁-C₂₀ acids and of C₁-C₈ alcohols, dimethoxyethane, diethoxyethane, C₁₀-C₃₀ fatty alcohols, C₁₀-C₃₀ fatty acids, C₁₀-C₃₀ fatty amides, C₁₀-C₃₀ fatty alkyl esters, and mixtures thereof.

15. The method according to claim 1, wherein the composition further comprises at least one polymerization inhibitor.

16. The method according to claim 15, wherein the at least one polymerization inhibitor is chosen from anionic and free-radical polymerization inhibitors.

17. The method according to claim 15, wherein the at least one polymerization inhibitor is chosen from sulfur dioxide, nitric oxide, lactone, boron trifluoride, hydroquinone and derivatives thereof, tert-butylhydroquinone, benzoquinone and derivatives thereof, catechol and derivatives thereof, anisole and derivatives thereof, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans, 3-sulfonene, and mixtures thereof.

18. The method according to claim 15, wherein the at least one polymerization inhibitor is present in the composition in an amount ranging from 10 ppm to 20% by weight relative to the total weight of the composition.

19. The method according to claim 18, wherein the inhibitor is present in an amount ranging from 10 ppm to 1% by weight relative to the total weight of the composition.

20. The method according to claim 1, wherein the microparticles or nanoparticles are chosen from mineral, organic, and mixed particles.

21. The method according to claim 20, wherein the microparticles or nanoparticles are metallic particles.

22. The method according to claim 21, wherein the metal of the metallic particles is chosen from alkali metals, alkaline-earth metals, transition metals, rare-earth metals, and alloys of these metals.

23. The method according to claim 22, wherein the metal is chosen from aluminum, copper, cadmium, selenium, silver, gold, indium, iron, platinum, nickel, molybdenum, silicon, titanium, tungsten, antimony, palladium, zinc, tin, and alloys thereof.

24. The method according to claim 23, wherein the metal is chosen from gold, silver, palladium, platinum, cadmium, selenium, and alloys thereof.

25. The method according to claim 20, wherein the microparticles or nanoparticles are mineral particles chosen from oxides, carbides, nitrides, borides, sulfides and hydroxides, and mineral salts.

26. The method according to claim 20, wherein the microparticles or nanoparticles are mineral particles chosen from clays, silicates, alumina, silica, kaolin, and hydroxyapatite.

27. The method according to claim 26, wherein the mineral particles are silica microbeads coated with polymethylhydrogenosiloxane.

28. The method according to claim 20, wherein the microparticles or nanoparticles are organic particles chosen from nylon powders, polyethylene powders, poly-β-alanine powders, polyfluorinated powders, acrylic copolymer powders, polystyrene powders, polyester powders, expanded microspheres made of thermoplastic material, silicone resin microbeads, metal soaps derived from organic carboxylic acids having from 8 to 22 carbon atoms, powders of synthetic hydrophilic polymers, acrylic polyamides, insoluble polyurethanes, and porous cellulose microspheres.

29. The method according to claim 1, wherein the microparticles or nanoparticles are in the form of nanotubes.

30. The method according to claim 29, wherein the nanotubes comprise at least one element belonging to groups IIA, IIIA, IVA, VA, VIII, IB, IIB, IIIB, VIB and VIIB of the Periodic Table of the Elements.

31. The method according to claim 30, wherein the nanotubes comprise at least one element belonging to group IVA.

32. The method according to claim 31, wherein the nanotubes comprise carbon.

33. The method according to claim 1, wherein the nanoparticles are chosen from luminescent semiconductive nanoparticles comprising at least one metal chosen from Zn, Cd and Hg and at least one metal chosen from S, Se and Te.

34. The method according to claim 33, wherein the nanoparticles comprise cadmium selenide or cadmium sulfide.

35. The method according to claim 1, wherein the microparticles or nanoparticles are chosen from compounds capable of swelling under the action of heat.

36. The method according to claim 35, wherein the compound capable of swelling under the action of heat is in the form of heat-expandable particles.

37. The method according to claim 36, wherein the particles are hollow particles comprising a cavity and a continuous envelope comprising at least one polymer chosen from vinylidene chloride/acrylonitrile/methyl methacrylate polymers, acrylonitrile/methyl methacrylate polymers, and acrylonitrile homopolymers.

38. The method according to claim 1, wherein the microparticles or nanoparticles are present in the composition in an amount ranging from 0.0001 to 30% by weight relative to the total weight of the composition.

39. The method according to claim 38, wherein the microparticles or nanoparticles are present in the composition in an amount ranging from 0.01 to 10% by weight relative to the total weight of the composition.

40. The method according to claim 1, wherein the composition further comprises at least one agent chosen from reducing agents, fatty substances, plasticizers, softeners, antifoams, moisturizers, pigments, clays, mineral fillers, UV-screening agents, mineral colloids, peptizers, solubilizing agents, fragrances, preserving agents, anionic surfactants, cationic surfactants, nonionic surfactants, amphoteric surfactants, fixing polymers, non-fixing polymers, polyols, proteins, vitamins, direct dyes, oxidation dyes, nacreous agents, mineral thickeners, and organic thickeners.

41. The method according to claim 40, wherein the at least one agent is encapsulated.

42. The method according to claim 1, wherein the composition is in the form of a lotion, a spray, or a mousse.

43. A method for cosmetically treating keratin fibers comprising applying to keratin fibers a composition comprising, in a cosmetically acceptable medium, at least one electrophilic monomer and microparticles or nanoparticles.

44. A method for reinforcing keratin materials comprising applying to keratin materials a composition comprising, in a cosmetically acceptable medium, at least one electrophilic monomer and microparticles or nanoparticles.

45. The method according to claim 44, wherein the keratin materials are keratin fibers.

46. The method according to claim 44, wherein the keratin materials are nails.

47. A cosmetic composition comprising, in a cosmetically acceptable medium, at least one electrophilic monomer and microparticles or nanoparticles other than gold or silver particles.

48. The composition according to claim 47, wherein the microparticles or nanoparticles are chosen from metal oxides, polymer particles, quantum dots, nanotubes, and nanofibrils.

49. The composition according to claim 47, wherein the microparticles or nanoparticles are not exclusively metallic.

50. A process for treating keratin materials, comprising:
applying at least one electrophilic monomer to the keratin materials, and
applying microparticles or nanoparticles to the keratin materials,
wherein said microparticles or nanoparticles are applied to the keratin materials either before or after applying the at least one electrophilic monomer.

51. A process for treating keratin materials, comprising:
applying to keratin materials, in the presence of at least one nucleophilic agent, a composition comprising, in a cosmetically acceptable medium, microparticles or nanoparticles and at least one electrophilic monomer.

52. The process according to claim 51, wherein the at least one nucleophilic agent is chosen from molecular compounds, oligomers, dendrimers, polymers comprising nucleophilic functions chosen from: R₂N—, NH₂—, Ph₃C—, R₃C—, PhNH—, pyridine, ArS—, R—C≡C—, RS—, HS—, RO—, R₂NH, ArO—, N₃—, OH—, ArNH₂, NH₃, I—, Br—, Cl—, RCOO—, SCN—, ROH, RSH, NCO—, CN—, NO₃—, ClO₄—, and water, wherein Ph is a phenyl group, Ar is an aryl group, and R is a C₁-C₁₀ aryl group.

53. The process according to claim 52, wherein the at least one nucleophilic agent is water.

54. The process according to claim 51, wherein the composition is applied to keratin materials that have been moistened beforehand with an aqueous solution having a pH that has been adjusted using a base, an acid, or an acid/base mixture.

55. The process according to claim 51, wherein the keratin materials are preimpregnated using a nucleophilic agent other than water.

56. The process according to claim 51, comprising reducing the keratin materials before applying the composition.

57. The process according to claim 56, wherein the keratin materials are reduced using a reducing agent chosen from anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, and monoethanolamine thioglycolate.

58. The process according to claim 51, wherein the composition further comprises a polymer chosen from poly(methyl methacrylate) and cyanoacrylate-based copolymers.

59. The process according to claim 51, further comprising rinsing.

60. A kit comprising
a first composition comprising at least one electrophilic monomer and optionally at least one anionic and/or free-radical polymerization inhibitor, and
a second composition comprising microparticles or nanoparticles in a cosmetically acceptable medium.
